(19) United States
(12) Patent Application Publication
(10) Pub. No.: US 2017/0252468 A1
(43) Pub. Date: Sep. 7, 2017

(54) COMPOSITE CARBON NANODOTS USED IN PHOTOACOUSTIC IMAGING, AND THEIR PREPARATION AND USE

(71) Applicant: Wenzhou Institute of Biomaterials and Engineering, Wenzhou (CN)

(72) Inventors: Zhe Liu, Wenzhou (CN); Qien Xu, Wenzhou (CN); Yuanhui Song, Wenzhou (CN); Yihong Li, Wenzhou (CN)

(21) Appl. No.: 15/057,129

(22) Filed: Mar. 1, 2016

(51) Int. Cl. A61K 49/22; C09K 11/06; C09K 11/02

(52) U.S. Cl. CPC A61K 49/225; C09K 11/02; C09K 11/06; C09K 2211/1037

(57) ABSTRACT

The present invention discloses a technique of preparation of composite nanodots, and their use in the field of photoacoustic imaging, wherein, the main components of the composition are carbon nanodots, and supporting component is methylene blue, and the composition has favourable biocompatibility characteristics with the average grain size of 150-300 nanometers, and the average zeta potential of −15 to 5 millivolts. The above said techniques for preparation of composite nanodots are safe, quick and simple, low cost, and easy to perform for industrialized production. Composite carbon nanodots have good biocompatibility and safety, high photoacoustic imaging sensitivity, and it is promising in gaining wider use in the fields of biomedical imaging, targeting diagnosis and therapy, drug screening and optimization, and in vivo labelling and tracing, and has potential value in personalized medicine.

### 1. TECHNICAL FIELD

The present invention relates to composite carbon nanodots used in photoacoustic imaging, and their preparation and use.

### 2. BACKGROUND OF THE INVENTION

Photoacoustic imaging (PAI) is a biomedical imaging technique newly developed in the last decade. It is a method for obtaining tomographic images and three dimensional images of biological organisms/tissues by means of an acoustic wave signal response (photoacoustic effect) delivered as a result of light rays generated by an excitation light ray irradiation medium. It combines the advantages of high sensitivity characteristics provided by optical imaging and deep penetration characteristics provided by acoustic imaging, and thus it can provide high resolution and high contrast imaging on deep tissues. As a result, it became one of the imaging modes with the highest potential of use. Since the biochemical substances from various sources in the bodies of living organisms, such as deoxyhemoglobins, oxyhemoglobins, melanins, oils and fats, and moisture content etc. can be stimulated by excitation lights of certain wave bands, and since these compounds are more closely associated with physiological functions, therefore, photoacoustic imaging can sensitively reflect physiological structures of organisms and provide abundant biological data about the anatomies, functions, metabolisms, molecules, and genes etc. of organisms. However, the scattering effect of light weakens the photoacoustic signal-to-noise ratio exponentially with the increasing depth of living tissues, and therefore, while performing imaging on relatively deep layers, the resolution becomes relatively low. Recently, research and development in photoacoustic contrast media is getting more and more attention. It is possible to transform acoustic and optical characteristics of local tissues and thus further improve photoacoustic imaging contrast and resolution by means of exogenous contrast media.

Currently, commonly seen photoacoustic contrast media comprise gold nanoparticles, single carbon nanotubes, and some other related nano-materials. These materials have relatively small particle size and good stability, but since their biocompatibility and biodegradability characteristics are rather poor, cytotoxicity levels are relatively high, and half-lives are relatively short, therefore their use in the field of photoacoustic imaging is limited. Moreover, photoacoustic probes based on near infrared fluorescent dyes and organic polymers have recently been a popular field of research, wherein methylene blue is a US Food and Drug Administration-approved (FDA) photoacoustic imaging dye. The absorption peak of methylene blue is near 664 nanometers, which is close to the infrared area, and which is also the fundamental reason of using methylene blue in photoacoustic imaging. Since the stabilities of these kinds of dyes are relatively low in the body, their metabolism cycles are short and insufficient, and they require a carrier to load the dye molecules for performing final clinical applications.

Carbon nanodots are attracting more and more attention due to their benefits such as chemical inertness, lack of optical scintillation, low photobleaching rates, low toxicity, and good biocompatibility. Compared to organic dyes and quantum dots that contain heavy metal ions, the carbon nanodots can be used in various fields, such as bioimaging, photocatalysis, detection, lasers, LEDs, power storage, and transformation devices. Recent developments in the field of carbon nanodot synthesis methods would enable top-down production method via improved carbon structures (such as graphene, multi-walled carbon nanotubes) or down-top production method via chemical substances comprising carbon (such as ammonium citrate and EDTA). It is noteworthy that, the carbon nanodots obtained with these methods all require surface oxidation or purification, so that they can be luminous and water-soluble. Besides, carbon nanodots obtained with a single-step method as well as having surface purification have also been reported. In these kinds of methods, microwave synthesis is particularly outstanding, and they have the benefits of quick initiation, easy control of heating, and homogeneous heating process. As a result, composite carbon nanodots prepared by simple, quick, and high-transformation rate microwave reaction methods have wide use prospects in photoacoustic imaging contrast material development, photoacoustic imaging micromanipulation, and photoacoustic perspective assisted surgery etc. fields.

### SUMMARY OF THE INVENTION

The present invention relates to providing composite nanodots based on carbon nanodots in order to overcome the shortcomings and deficiencies of the prior art. The nano-composite photoacoustic contrast medium has favourable biocompatibility and safety characteristics. It has high photoacoustic imaging sensitivity and it is promising in gaining wider use in the fields of biomedical imaging, targeting diagnosis and therapy, drug screening and optimization, and in vivo labelling and tracing. Moreover, it has potential value in the field of personalized medicine.

The present invention provides a safe, quick, simple, and low cost single-step method for preparing water-soluble multicolour carbon nanodots via microwave radiation and a highly effective separation and purification method thereof, and thus load the obtained multicolour carbon nanodots on methylene blue molecules in order to ensure high-sensitivity photoacoustic imaging. The above said techniques for preparation and purification of composite nanodots based on carbon nanodots are safe, quick and simple, low cost, and easy to perform industrialized production.

In order to achieve the first purpose of the present invention, the present invention aims to provide photoacoustic contrast media with the main components of carbon nanodots that can be loaded on tissues. Moreover, carbon sources such as wolfberry leaching agent, soy milk, and dietary milk used in preparation of carbon nanodots are added, methylene blue is loaded on tissues, and the average grain size is 150-300 nanometers, while the average zeta potential is −15 to 5 millivolts.

In order to achieve the second purpose of the invention, implementation of the present invention at least involves inventive steps as follows:
a. forming a solution of the above said carbon sources and 0.5 milligrams/millilitres of methylene blue by means of mixing in the ratio of 0.1:1 to 10:1 by volume, diluting the mixture 1-100 folds with ultra-pure water, and thus obtaining a precursor solution;
b. placing the above said precursor solution in a microwave reaction instrument, and setting the instrument parameters as follows: Temperature: 100-180° C., time: 15-300 minutes;
c. The reaction system is allowed to wait for 30 minutes, a clear solution is obtained as a result of centrifugal separation, and crude product of composite nanodots is obtained;

Purification is made on the above said crude product of composite nanodots using ultrafiltration or dialysis method, and thus obtaining composite nanodots that can be used in photoacoustic imaging.

The novel composite nanodots developed with the present invention have practical significance in the fields of developing photoacoustic contrast media used in medicine, expanding the preparation techniques of photoacoustic contrast media, and in general applicability of photoacoustic imaging in the field of biomedicine. The method of water soluble, multicolour carbon nanodot preparation via microwave irradiation according to the present invention is completely performed in aqueous solution, is easy, quick and simple, has low toxicity, and its raw materials are easy to obtain. The water-soluble, multicolour carbon nanodots obtained after dialysis or ultrafiltration purification have beneficial monodispersion, good stability, good water solubility, and can be widely used as fluorescent marker in biological detection and analysis.

The present invention provides use of composite nanodots that are loaded on methylene blue in the field of photoacoustic imaging. In photoacoustic field, MDA MB231 nude mice are anesthesized with isoflurane, and then intravenously injected with 150 microliters of photoacoustic composite carbon nanodot probe specimen through their tails. Afterwards, photoacoustic imaging is performed on nude mice at different time points, and it was found that, at the tumor place, the composite carbon nanodots have generated light and acoustic signals when stimulated with a near infrared light wavelength of 640 nanometers, which clearly indicates that the tumor arteries and tissues are concentrated with composite carbon nanodots, and after 6 hours following injection, their basic metabolism is completed through urinary bladder. The reconstructed graph of the photoacoustic signals collected by probes are shown in FIG. 4. Conclusion: Composite nanodots loaded with methylene blue can significantly improve photoacoustic imaging contrast and resolution in small animals, and therefore, they are expected to have wide usage prospects in the field of biomedical imaging.

The figures attached to the specification and the below given specific embodiment are given for the purpose of describing the invention better.

### BRIEF DESCRIPTION OF THE SEVERAL VIEWS OF THE DRAWINGS

FIG. 1 is; a flow chart of the preparation technique for the composite carbon nanodots of the present invention;

FIG. 2a-2c are; the particle size distribution and zeta potential phenograms of the composite carbon nanodots of the present invention;

FIG. 3a-3c are; the fluorescence spectogram of the composite carbon nanodots of the present invention at the excitation wavelength of 340-440 nanometers (left side) and at the excitation wavelength of 650 nanometers (right side);

FIG. 4 is; the reconstructed graph of the photoacoustic signals collected by probes, wherein the composite carbon nanodots of embodiment 1 have generated light and acoustic signals when stimulated with a near infrared light excitation on small animals via intravenous injection.

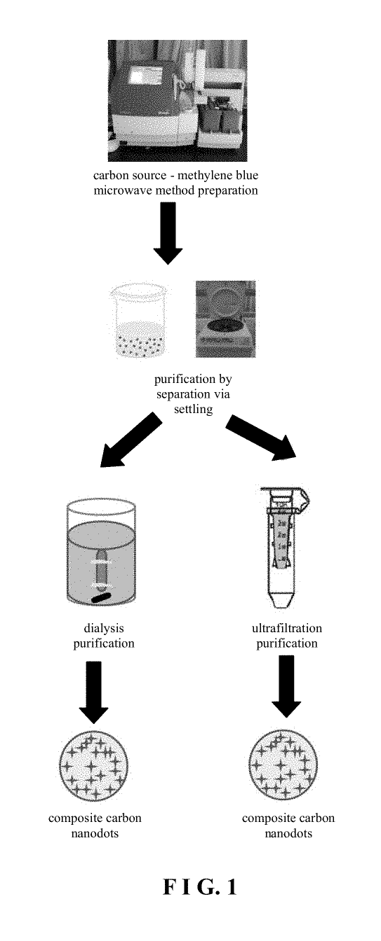

### DETAILED DESCRIPTION OF THE EMBODIMENTS OF THE INVENTION

Below given specific descriptions about the present invention through embodiments are only for better understanding of the invention, and do not form any limitation in the protection scope of the invention disclosed in the claims, and persons skilled in the related technical field can make some non-essential changes and modifications on the present invention according to the contents of the above said description.

The raw materials used in the preparation of the present invention are all commercially available.

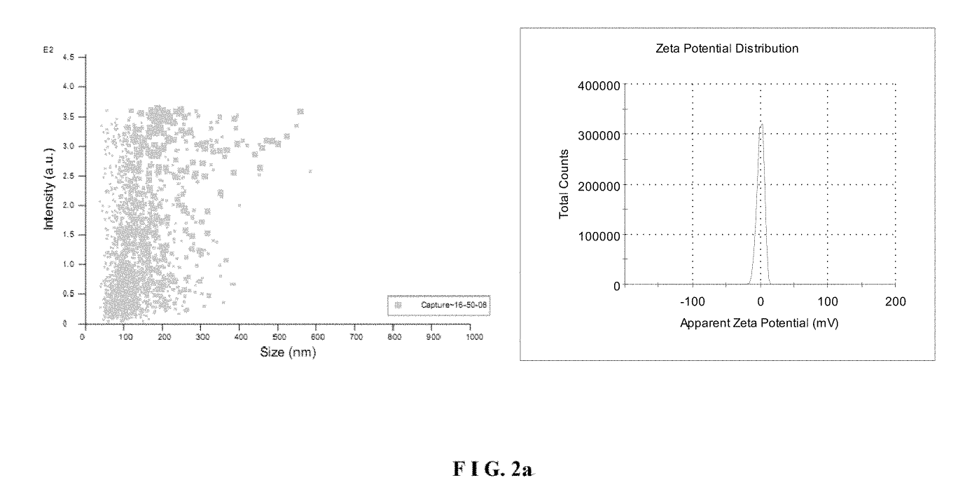

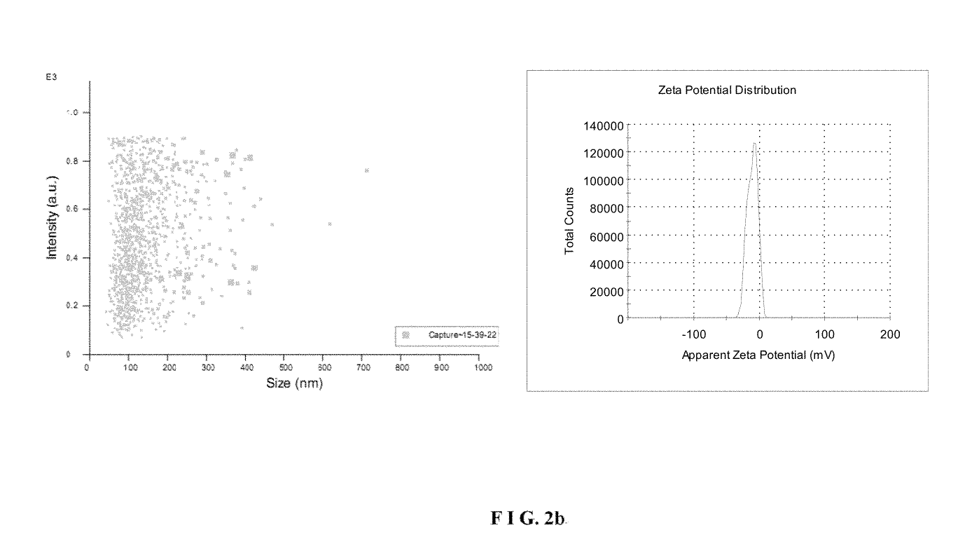

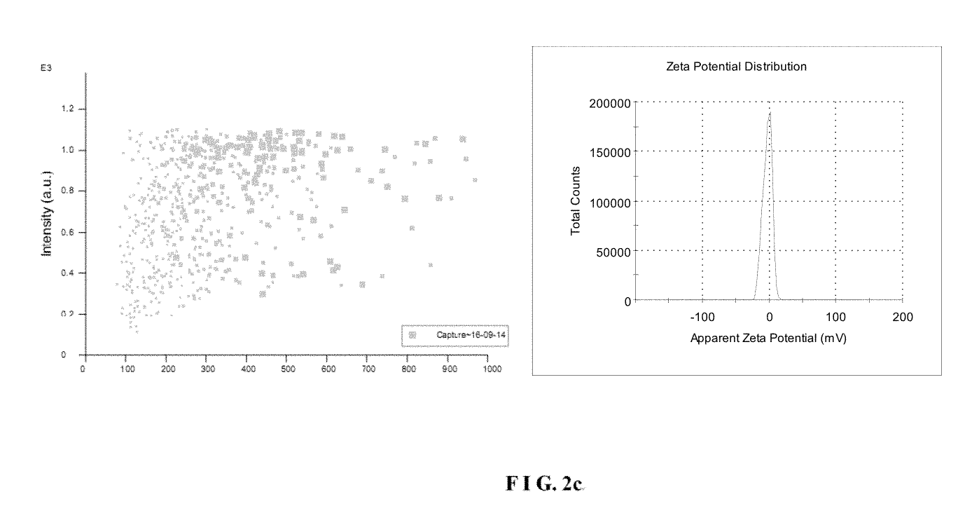

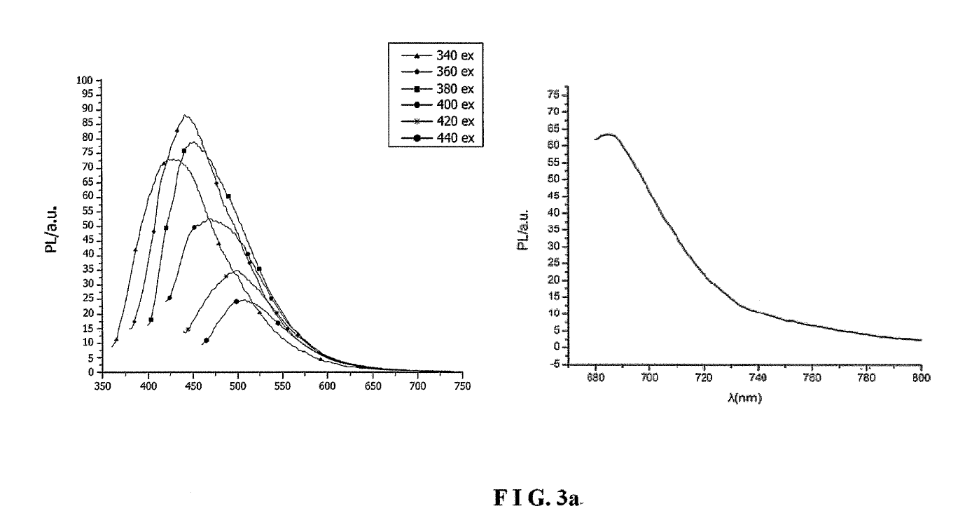

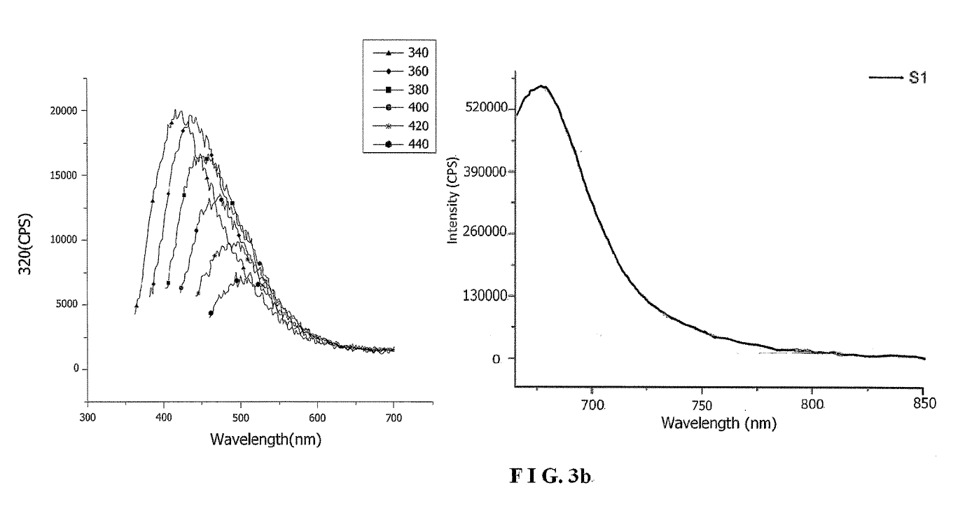

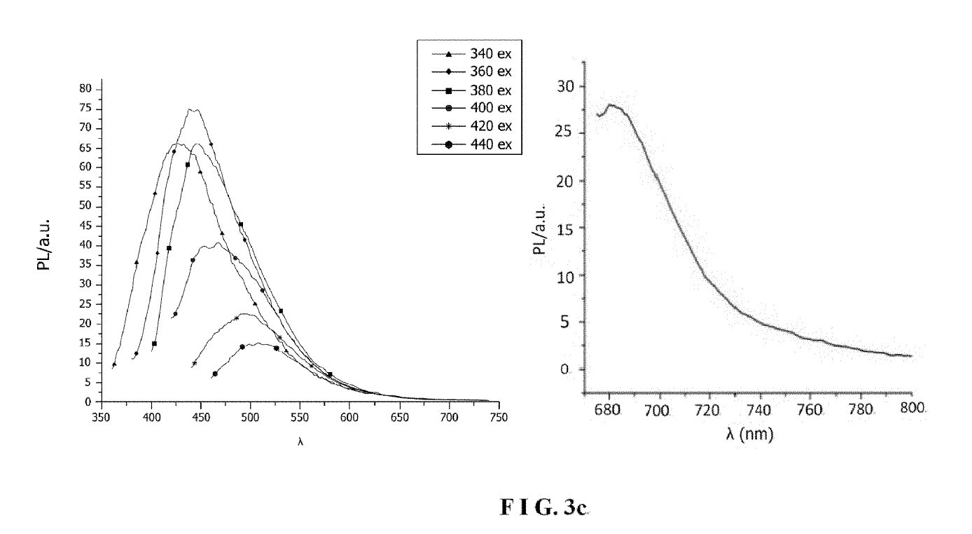

Embodiment 1: 2 milliliters of wolfberry leaching agent and 2 milliliters of methylene blue solution (0.5 milligrams/milliliters) are mixed, and then diluted 1 fold using ultra-pure water to obtain a precursor solution. The precursor solution is then placed in a 5 milliliter special glass bottle for use in microwave reaction instruments, and afterwards, the bottle is placed in a microwave reaction instrument, and the reaction conditions are set to 180 degrees centigrade and 30 minutes. The reaction system is allowed to wait for 50 minutes, and then centrifugated, and the supernatant liquid is retained, and composite carbon nanodots are obtained following ultrafiltration. Measurements are made by a laser particle analyzer according to dynamic light scattering principle, and the composite carbon nanodot grain size distribution are found as 179±77.1 nanometers (FIG. 2a, left), while the surface zeta potential is found as 0.85±5.29 millivolts (FIG. 2a, right). The fluorescence properties of composite carbon nanodots detected by a fluorescence detector device are shown in FIG. 3a.

Embodiment 2: 1 milliliter of soy milk and 10 milliliters of methylene blue solution (0.5 milligram/milliliter) are mixed, and then diluted 5 folds using ultra-pure water to obtain a precursor solution. 5 milliliters of obtained precursor solution is then placed in a 5 milliliter special glass bottle for use in microwave reaction instruments, and afterwards, the bottle is placed in a microwave reaction instrument, and the reaction conditions are set to 100 degrees centigrade and 5 hours. The photoacoustic contrast medium is reacted for 1 hour, and then centrifugated, and the supernatant liquid is retained, and composite carbon nanodots are obtained following dialysis. Measurements are made by a laser particle analyzer according to dynamic light scattering principle, and the grain size distribution of the composite carbon nanodots loaded with methylene blue are found as 191±107 nanometers (FIG. 2b, left), while the surface zeta potential is found as −10.2±8.0 millivolts (FIG. 2b, right). The fluorescence properties of composite carbon nanodots detected by a fluorescence detector device are shown in FIG. 3b.

Embodiment 3: 10 milliliters of freshly brewed milk and 1 milliliter of methylene blue solution (0.5 milligram/milliliter) are mixed, and then diluted 10 folds using ultra-pure water to obtain a precursor solution. 5 milliliters of obtained precursor solution is then placed in a 5 milliliter special glass bottle for use in microwave reaction instruments, and afterwards, the bottle is placed in a microwave reaction instrument, and the reaction conditions are set to 160 degrees centigrade and 2 hours. The photoacoustic contrast medium is reacted for 1 hour, and then centrifugated, and the supernatant liquid is retained, and composite carbon nanodots are obtained following ultrafiltration. Measurements are made by a laser particle analyzer according to dynamic light scattering principle, and the grain size distribution of the composite carbon nanodots loaded with methylene blue are found as 264±174 nanometers (FIG. 2c, left), while the surface zeta potential is found as −2.42±6.67 millivolts (FIG. 2c, right). The fluorescence properties of composite carbon nanodots detected by a fluorescence detector device are shown in FIG. 3c.

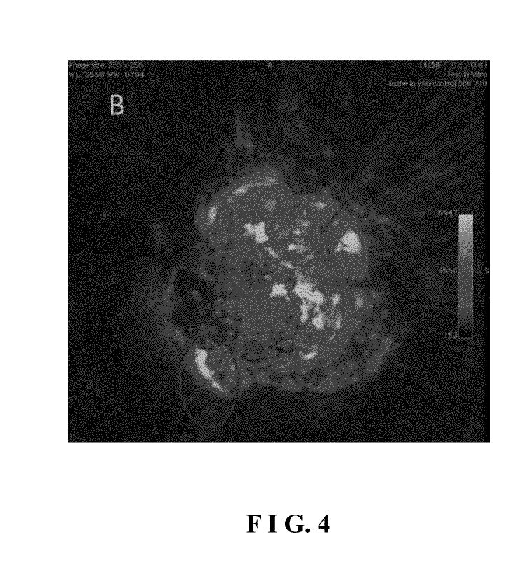

In photoacoustic field, MDA MB231 nude mice are anesthesized with isoflurane, and then intravenously injected with 150 microliters of photoacoustic composite carbon nanodot probe specimen through their tails. Afterwards, photoacoustic imaging is performed on nude mice at different time points, and it was found that, at the tumor place, the composite carbon nanodots have generated light and acoustic signals when stimulated with a near infrared light wavelength of 640 nanometers, which clearly indicates that the tumor arteries and tissues are concentrated with composite carbon nanodots, and after 6 hours following injection, their basic metabolism is completed through urinary bladder. The reconstructed graph of the photoacoustic signals collected by probes is shown in FIG. 4. Conclusion: Composite nanodots loaded with methylene blue can significantly improve photoacoustic imaging contrast and resolution in small animals, and therefore, they are expected to have wide usage prospects in the field of biomedical imaging.

What is claimed is:

1. Composite carbon nanodots used in photoacoustic imaging, characterized in that; the main components of the composition are carbon nanodots, and methylene blue is used as a support ligand.

2. The composite carbon nanodots used in photoacoustic imaging according to claim 1, characterized in that; the carbon sources of the carbon nanodots in the composite nanodots are any of wolfberry leaching agents, soy milk, or dietary milk.

3. The composite carbon nanodots used in photoacoustic imaging according to claim 1, characterized in that; the average grain size of the composite nanodots is 150-300 nanometers, and the average zeta potential is −15 to 5 millivolts.

4. A preparation method of composite carbon nanodots used in photoacoustic imaging according to claim 1, characterized in that; it comprises the operation steps of:
a. forming a solution of the above said carbon sources and 0.5 milligrams/millilitres of methylene blue by means of mixing in the ratio of 0.1:1 to 10:1 by volume, diluting the mixture 1-10 folds with ultra-pure water, and thus obtaining a precursor solution;
b. placing the above said precursor solution in a microwave reaction instrument, and setting the instrument parameters as follows: Temperature: 100-180° C., time: 30-300 minutes;
c. reacting the system for more than 30 minutes, obtaining a clear solution as a result of centrifugal separation, and obtaining a crude product of composite nanodots.

5. The preparation method of composite carbon nanodots used in photoacoustic imaging according to claim 4, characterized in that; it also comprises application of purification on the crude product of composite nanodots using dialysis method.

6. The preparation method of composite carbon nanodots used in photoacoustic imaging according to claim 4, characterized in that; it also comprises application of purification on the crude product of composite nanodots using ultrafiltration method.

7. The use of the composite carbon nanodots used in photoacoustic imaging according to claim 1 in the field of photoacoustic imaging.

8. The use of the composite carbon nanodots used in photoacoustic imaging according to claim 2 in the field of photoacoustic imaging.

9. The use of the composite carbon nanodots used in photoacoustic imaging according to claim 3 in the field of photoacoustic imaging.
